(19) United States
(12) Patent Application Publication
(10) Pub. No.: US 2011/0256559 A1
(43) Pub. Date: Oct. 20, 2011

(54) Method for Detecting Soluble Amyloid Precursor Protein (APP) Alpha and/or Soluble APP Beta

(75) Inventors: Bernadette Allinquant, Paris (FR); Christiane Rose, Paris (FR)

(73) Assignee: INSERM (Institut National de la Sante et de la Recherche Medicale), Paris (FR)

(21) Appl. No.: 13/124,234

(22) PCT Filed: Oct. 19, 2009
(86) PCT No.: PCT/EP2009/063643

§ 371 Date: Jun. 30, 2011

(30) Foreign Application Priority Data
Oct. 20, 2008 (EP) 08305704.2

(51) Int. Cl. G01N 33/566

(52) U.S. Cl. 435/7.92; 436/501; 435/7.1

(57) ABSTRACT

The present invention relates to a method for detection or quantification of sAPPα and/or sAPPβ in a sample and their use for diagnosing and/or monitoring a neurodegenerative disease in a subject in need thereof and kit for use in said method.

### FIELD OF THE INVENTION

The invention relates to methods for the detection or quantification of sAPPα and/or sAPPβ in a sample and their use for diagnosing and/or monitoring a neurodegenerative disease.

### BACKGROUND OF THE INVENTION

Alzheimer's disease is a progressive neurodegenerative disease characterized by progressive memory deficits, impaired cognitive function, altered and inappropriate behavior, and a progressive decline in language function. It is the most prevalent age-related dementia, affecting an estimated 18 million people worldwide, according to the World Health Organization. As medical advances continue to prolong the human lifespan, it is certain that Alzheimer's disease will affect an increasing proportion of the population. There is no cure for Alzheimer's disease, and current therapies provide only temporary and symptomatic relief, while doing little to counteract disease progression. Pathologically, Alzheimer's disease patients display cortical atrophy, loss of neurons and synapses, and hallmark extracellular senile plaques and intracellular neurofibrillary tangles. Senile (or neuritic) plaques are composed of aggregated amyloid β-peptide (Aβ), which results from APP (Amyloid Precursor Protein) cleavage, and are found in large numbers in the limbic and association cortices. It is widely hypothesized that the extracellular accumulation of Aβ contributes to axonal and dendritic injury and subsequent neuronal death. Neurofibrillary tangles consist of pairs of about 10 nm filaments wound into helices (paired helical filaments or PHF). Immunohistochemical and biochemical analysis of neurofibrillary tangles revealed that they are composed of a hyperphosphorylated form of the microtubule-associated protein tau. These two classical pathological lesions of Alzheimer's disease can occur independently of each other. However, there is growing evidence that the gradual accumulation of Aβ and Aβ-associated molecules leads to the formation of neurofibrillary tangles. As such, much research is directed at inhibiting the generation of the amyloid β-peptide.

Amyloid β peptide is formed by the amyloidogenic processing of APP. This processing requires cleavage at two distinct sites by the β-secretase and γ-secretase. BACE1 (β-site APP cleavage enzyme 1) was identified as the major β-secretase and cleaves APP to release an ecto-domain: sAPPβ, into the extracellular space. The remaining C-terminal fragment undergoes subsequent cleavage by γ-secretase to release Aβ and the APP intracellular C-terminal domain (AICD).

In non-pathological situation, APP is predominantly cleaved by the α-secretase within the Aβ domain, thereby precluding the generation of Aβ. This cleavage releases a soluble form of APP: sAPPα, which seems to have neuroprotective functions. Subsequent cleavage of the 83-amino acid remaining C-terminal fragment (C83) releases p3, which is non-amyloidgenic, and the AICD. The functions of these fragments are not known.

As much research is directed at inhibiting the generation of the amyloid β-peptide, various therapeutic approaches, based on inhibition of BACE1 and γ-secretase or activation of the α-secretase pathway, are currently under investigation.

Accordingly, there is a need to develop efficient methods and tools for measuring the cleavage products of APP, such as sAPPβ and sAPPα, in a sample. Such methods and tools may for example be used to identify new therapeutic molecules that inhibits BACE1 and γ-secretase or activates the α-secretase pathway, or to monitor the efficacy of a therapeutic treatment.

In this context, such methods and tools have already been disclosed: for example, WO2008/008463 describes an antibody that selectively binds to sAPPβ but not to sAPPα. Known antibodies, that bind APP and sAPPα or that bind selectively sAPPα, have also been disclosed in WO2008/009868. In addition, WO2008/009868 describes an advantageous method for measuring sAPPα in a sample, which comprises a first step of thermal treatment of the sample. Said thermal treatment is meant to allow certain specific epitopes of sAPPα to be more accessible to antibodies.

In addition, Lewczuk et al. demonstrated a key role of sAPPα and sAPPβ as biomarkers of Alzeihmer's disease.

Therefore, there is a need for a more accurate assay for measuring sAPPα and/or sAPPβ in a sample.

### SUMMARY OF THE INVENTION

The invention relates to a method for detecting or quantifying the presence of sAPPα and/or sAPPβ in a sample, said method comprising:
treating the sample with a disulfide bonds reducing agent,
measuring sAPPα or sAPPβ in said sample by immunological detection.

In a preferred embodiment, the method of the invention is a method for detecting or quantifying the presence of sAPPα in a sample.

In one embodiment of the invention, said disulfide bonds reducing agent is 2-mercaptoethanol (ME), dithiothreitol (DTT), tris(2-carboxyethyl)phosphine (TCEP) or a combination thereof.

In one embodiment of the invention, said sample is a culture medium sample, a whole blood sample, a serum sample, a plasma sample, a cerebrospinal fluid sample, or a brain tissue sample.

In one embodiment of the invention, the immunological detection of sAPPα and/or sAPPβ is carried out by using at least one antibody that binds specifically to sAPPα or sAPPβ.

In one embodiment of the invention, the immunological detection of sAPPα and/or sAPPβ is carried out by an enzyme immunoassay or enzyme-linked immunoassay (EIA or ELISA). In another embodiment of the invention, the immunological detection of sAPPα and/or sAPPβ is carried out by homogeneous time resolved fluorescence (HTRF).

In one embodiment, a second antibody that binds to an epitope in the N-terminal domain of sAPPα or sAPPβ is used to carry out the immunological detection of sAPPα and/or sAPPβ.

The invention relates to a method for determining and/or monitoring a neurodegenerative disorder in a subject in need thereof, said method comprising quantifying sAPPα and/or sAPPβ in a sample obtained from said subject according to the method as described here above.

In one embodiment, said method is for diagnosing and/or monitoring a neurodegenerative disorder, said neurodegenerative disorder being Alzheimer's disease, early onset familial Alzheimer's disease, amyotrophic lateral sclerosis (Lou Gehrig's disease), Binswanger's disease, corticobasal degeneration (CBD), dementia lacking distinctive histopathology (DLDL), frontotemporal dementia (FTD), Huntington's chorea, multiple sclerosis, myasthenia gravis, Parkinson's disease, trisomy 21 or progressive supranuclear palsy (PSP).

The invention relates to a kit for use in the methods as described here above, comprising as separate components:
a disulfide bonds reducing agent, and
at least one antibody that binds specifically to sAPPα or sAPPβ.

In one embodiment, said kit further comprises a second antibody that binds to an epitope in the N-terminal domain of sAPPα or sAPPβ.

### DETAILED DESCRIPTION OF THE INVENTION

The inventors made the observation that sAPPα and sAPPβ are dimerized through disulfide bridges. This observation led them find that conventional assays based on immunological detection of sAPPα or sAPPβ are therefore inefficient to detect or correctly measure sAPPα or sAPPβ in a sample.

The invention thus relates to an improved method for measuring the presence of sAPPα and/or sAPPβ in a sample, said method comprising:
treating the sample with a disulfide bonds reducing agent,
measuring sAPPα and/or sAPPβ in said sample by immunological detection.

### DEFINITIONS

As used herein, the term “sAPPα” corresponds to a 100-120 kDa protein (1-687 amino acids of APP770, 1-668 of APP751, 1-612 of APP695), obtained by cleavage of APP with α-secretase between amino acid Lys(16) and Leu(17) of the Aβ region.

As used herein, the term “sAPPβ” corresponds to a 98-118 kDa protein, obtained by cleavage of APP with β-secretase between amino acid Met 671 and Asp 672 of APP770, Met 652 and Asp 653 of APP 751, Met 596 and Asp 597 of APP 695.

As used herein, the term “disulfide bonds reducing agent” refers to an agent that is capable to reduce disulfide bonds such as 2-mercaptoethanol (ME), dithiothreitol (DTT) or tris(2-carboxyethyl)phosphine (TCEP).

As used herein, the term “subject” denotes a mammal, such as a rodent, a feline, a canine, and a primate. Preferably a subject according to the invention is a human.

As used herein, the term “antibody” refers to a protein capable of specifically binding an antigen, typically and preferably by binding an epitope or antigenic determinant or said antigen. The term “antibody” also includes recombinant proteins comprising the binding domains, as well as variants and fragments of antibodies. Examples of fragments of antibodies include Fv, Fab, Fab′, F(ab′)2, dsFv, scFv, sc(Fv)2, diabodies and multispecific antibodies formed from antibody fragments.

As used herein, “measuring” encompasses detecting or quantifying. As used herein, “detecting” means determining if sAPPα or sAPPβ is present or not in a sample and “quantifying” means determining the amount of sAPPα or sAPPβ in a sample.

### THE INVENTION

One object of the invention is a method for detecting or quantifying the presence of sAPPα and/or sAPPβ in a sample, said method comprising:
treating the sample with a disulfide bonds reducing agent,
measuring sAPPα and/or sAPPβ in said sample by immunological detection.

Said method provides a more efficient detection of sAPPα and/or sAPPβ in a sample compared to conventional methods based on immunological detection and compared to the method described in WO2008/009868 (see examples).

In a preferred embodiment, the method of the invention is a method for detecting or quantifying the presence of sAPPα in a sample.

In one embodiment of the invention, said disulfide bonds reducing agent is 2-mercaptoethanol (ME), dithiothreitol (DTT), or tris(2-carboxyethyl)phosphine (TCEP) or a combination thereof.

In another embodiment of the invention, said disulfide bonds reducing agent is dithiothreitol (DTT).

In one embodiment of the invention, the sample is treated with a disulfide bonds reducing agent during 10 to 45 min, preferably during, 15 to 35 min, more preferably during 20 to 30 min.

In one embodiment of the invention, the sample is treated with 1 to 10 mM of disulfide bonds reducing reagent, preferably with 5 to 10 mM of disulfide bonds reagent.

In one embodiment of the invention, the sample is treated with a disulfide bonds reducing agent on ice.

In one embodiment of the invention, the sample is treated with 10 mM of DTT during 30 min on ice.

According to the invention, the sample susceptible to contain sAPPα and/or sAPPβ is a biological sample, such as cell lysates or culture medium, or is a body fluid such as serum, plasma, or cerebrospinal fluid, brain tissue (cortex, hippocampus, striatum, brainstem, cerebellum). While brain tissue is used, it is homogenized in absence of detergent, preferably for example in the presence of Tris buffer saline.

In one embodiment, said sample is a cerebrospinal fluid.

In another embodiment, said sample is a serum sample or a plasma sample.

In another embodiment, said sample is a conditioned culture medium such as conditioned culture medium from SH-SY5Y neuroblastoma cell line treated or not with drugs such as PdBu (activator of α-secretase pathway). As used herein, “conditioned culture medium” refers to culture medium in which cells were cultivated and optionally treated with a drug, such as for example a pharmacological drug. A “conditioned culture medium” is not a fresh culture medium.

In another embodiment, said sample is a conditioned culture medium such as conditioned culture medium from primary mouse cortical neurons treated or not with drugs such as PACAP or EGCG (two other activators of alpha secretase activity).

According to the invention, the immunological detection or quantification of sAPPα and/or sAPPβ is achieved by any methods known in the art using at least one antibody that binds specifically to sAPPα and/or sAPPβ.

Examples of said methods include, but are not limited to, standard electrophoretic and immunodiagnostic techniques such as western blots, immuno-precipitation assay, radioimmunoassay, ELISA (enzyme-linked immunosorbant assay), “sandwich” immunoassay, immunoradiometric assay, gel diffusion precipitation reaction, immunodiffusion assay, precipitation reaction, agglutination assay (such as gel agglutination assay, hemagglutination assay, etc.), complement fixation assay, protein A assay, immunoelectrophoresis assay, high performance liquid chromatography, size exclusion chromatography, solid-phase affinity, etc.

In a preferred embodiment, the immunological detection of sAPPα and/or sAPPβ is not carried out by a method which is based on the migration of proteins, such as Western blot.

According to the invention, an antibody that binds specifically to sAPPα or sAPPβ is an antibody that does not cross react with each other.

In one embodiment, the antibody binds specifically to sAPPα.

Said antibody is for example an antibody that binds on the cleavage site of sAPPα.

In one embodiment, the antibody binds specifically to sAPPα and not to sAPPβ. Such an antibody is typically an antibody that binds to a fragment of sAPPα that is absent from sAPPβ, i.e. an antibody that recognizes an epitope located in the C-terminus of sAPPα, between residues 596 and 612 of APP695.

Examples of said antibody include, but are not limited to, the monoclonal antibody 6E10, the monoclonal antibody 2B3 and the rabbit antibody 3329 (PMID 9465092).

In another embodiment, the antibody binds specifically to sAPPβ.

Examples of said antibody include, but are not limited to, Signet's rabbit anti sAPPβ (specific for the soluble fragment cleaved n-terminus to the beta secretase cleavage site of amyloid precursor protein) purchased by Covance Research Products, the antibody disclosed in WO2008/008463, the polyclonal IgG rabbit antibody JP18957 purchased by IBL.

In another embodiment, the immunological detection or quantification of sAPPα and/or sAPPβ is achieved by any methods known in the art using at least one antibody that binds specifically to sAPPα or sAPPβ and at least one antibody that does not bind specifically to sAPPα or sAPPβ.

According to the invention, an antibody that does not bind specifically to sAPPα or sAPPβ is an antibody that binds to APP or Aβ for example. For example, said antibody is an antibody that binds to an epitope in the N-terminal domain of sAPPα or sAPPβ. In one embodiment, the antibody does not bind specifically to sAPPα or sAPPβ.

Examples of said antibody include, but are not limited to, the monoclonal antibody BAN50 (PMID: 10480887), the monoclonal antibody 22C11, the polyclonal antibody polyC11 (Upstate/Millipore, Cat AB5368, Chemicon), the polyclonal antibody sAPP from OYC (Cat APP-KPI-Antiserum), the polyclonal antibody sAPP from Signet Covance (Cat SIG-39139), the monoclonal antibody 1E8 (U.S. Pat. No. 6,849,416).

According to the invention, the antibody that binds to sAPPα or sAPPβ may be labelled with a detectable molecule or substance. Examples of suitable labels for this purpose include a chemiluminescent agent, a colorimetric agent, an energy transfer agent, an enzyme, a substrate of an enzymatic reaction, a fluorescent agent, or a radioisotope. The label may be coupled directly or indirectly by any known method in the art.

In one embodiment, the detection or quantification of sAPPα or sAPPβ in a sample may be achieved by a protein chip array system, wherein the antibody that binds to sAPPα or sAPPβ is coated directly or indirectly on a protein chip array. The sample to be tested is labelled by biotinylation in vitro. Biotinylated sAPPα or sAPPβ trapped on the array are then detected by avidin or streptavidin which strongly binds biotin. If avidin is conjugated with horseradish peroxidase or alkaline phosphatase, the captured sAPPα or sAPPβ can be visualized by enhanced chemical luminescence. The amount of protein bound to the antibody that binds to sAPPα or sAPPβ represents the level of sAPPα or sAPPβ in the sample. Other methods, like immunochemical staining, surface plasmon resonance, matrix-assisted laser desorption/ionization-time of flight, can also be used to detect the captured proteins.

In another embodiment of the invention, the detection or quantification of sAPPα and/or sAPPβ in a sample may be achieved by a cytometric bead array system wherein the antibody that binds to sAPPα or sAPPβ is coated directly or indirectly on beads.

In another embodiment of the invention, the detection or quantification of sAPPα and/or sAPPβ in a sample may be achieved by a competitive immunoassay. Example of competitive immunoassays include enzyme immunoassay or enzyme-linked immunoassay (EIA or ELISA), fluorescent immunoassay, radiometric or radioimmunoassay (RIA), magnetic separation assay (MSA), lateral flow assay, diffusion immunoassay, immunoprecipitation immunoassay.

In one example of competitive immunoassay, the quantification of sAPPα and/or sAPPβ is achieved by
combining a sample containing sAPPα and/or sAPPβ with a known amount of a labelled sAPPα to create a spiked sample,
binding labelled and unlabelled sAPPα and/or sAPPβ in the spiked sample with an antibody anti-sAPPα and/or anti-sAPPβ, wherein the antibody binds specifically to sAPPα or sAPPβ to create complexes and to labelled sAPPα or sAPPβ to create labelled complexes,
measuring the amount of labelled complex
calculating the amount of sAPPα and/or sAPPβ present in the sample.

In one embodiment, said antibody that binds to sAPPα or sAPPβ may be coated to a solid support, said solid support comprising a protein binding surface such as a microtiter plate, a colloid metal particle, an iron oxide particle, a latex particle or a polymeric bead.

In one embodiment, the labelled sAPPα or sAPPβ may comprise a label such as a chemiluminescent agent, a colorimetric agent, an energy transfer agent, an enzyme, a fluorescent agent, or a radioisotope. Examples of chemiluminescent agent include an enzyme that produces a chemiluminescent signal in the presence of a substrate(s) that produce chemiluminescent energy when reacted with the enzyme. Examples of such an enzyme include horseradish peroxidase (HRP) and alkaline phosphatase (AP). Other examples of a chemiluminescent agent include a non-enzymatic direct chemiluminescent label, such as Acrinidium ester system. Examples of a colorimetric agent include an enzyme such as horseradish peroxidase, alkaline phosphatase, and acetylcholine esterase (AChE). Examples of energy transfer agent include fluorescent lanthanide chelates. Examples of fluorescent agents include fluorescent dyes. Examples of radioisotopes include ¹²⁵I, ¹⁴C and ³H.

While the spiked sample is incubated with the antibody that binds to sAPPα or sAPPβ, the sAPPα present in the sample and the added labelled sAPPα or sAPPβ compete for binding to the antibody. Labelled sAPPα or sAPPβ will then be able to bind the antibody depending on the relative concentration of the unlabeled sAPPα or sAPPβ present in the sample. Thus, when the amount of labelled sAPPα or sAPPβ is measured, it is inversely proportional to the amount of unlabeled sAPPα or sAPPβ present in the sample. The amount of sAPPα or sAPPβ present in the sample may then be calculated based on the amount of labelled sAPPα or sAPPβ measured, using standard techniques.

In another example of competitive immunoassay, the quantification of sAPPα or sAPPβ is achieved by the antibody coupled with or conjugated with a ligand, said ligand binding to an additional antibody added to the sample. One example of said ligand is fluorescein. The additional antibody may be bound to a solid support. In this example of competitive immunoassay, the additional antibody binds to the ligand coupled with the antibody that binds in turn to (i) sAPPα or sAPPβ present in the sample and (ii) labelled sAPPα or sAPPβ added to the sample. Said mass complex formed allows isolation and measurement of the signal generated by the label coupled with the labelled sAPPα or sAPPβ.

In another example of competitive immunoassay, sAPPα or sAPPβ may be bound to a solid support, and incubated with (i) an antibody that binds to sAPPα or sAPPβ and (ii) a sample containing the sAPPα or sAPPβ to be measured. The antibody binds either the sAPPα or sAPPβ bound to the solid support or the sAPPα or sAPPβ present in the sample, in relative proportion depending of the concentration of the sAPPα or sAPPβ present in the sample. The antibody that binds to sAPPα or sAPPβ bound to the solid support is then bound to another antibody that is coupled with a label. The amount of signal generated from the label is then detected to measure the amount of sAPPα or sAPPβ. Such a measurement will be inversely proportional to the amount of sAPPα or sAPPβ present in the sample. Such an assay may be used in a microtiter plate.

In another example of competitive immunoassay, the sAPPα or sAPPβ to be measured compete with sAPPα or sAPPβ that is bound to a first solid support particle, such as Ficoll, for the antibody that is bound to or coated to a second solid support particle. Cross-binding or agglutination between the particles occurs and forms clumps of co-agglutination lattice. Alternatively, the antibody binds to the free sAPPα or sAPPβ in the sample, so that the amount of agglutination is inversely proportional to the amount of sAPPα or sAPPβ in the sample. The amount of agglutination may be measured using standard techniques, such as spectrophotometry.

In another embodiment of the invention, the quantification of sAPPα and/or sAPPβ in a sample may be achieved by a non-competitive immunoassay referred as immunometric, “two-site” or “sandwich” immunoassays, wherein the sAPPα or sAPPβ may be bound to or sandwiched between two antibodies that bind to sAPPα or sAPPβ.

Examples of non-competitive immunoassays include enzyme immunoassay or enzyme-linked immunoassay (EIA or ELISA), fluorescent immunoassay, radiometric or radioimmunoassay (RIA), magnetic separation assay (MSA), lateral flow assay, diffusion immunoassay, immunoprecipitation immunoassay, immunosorbent or “antigen-down” assay using antibodies that bind to sAPPα or sAPPβ bound to a solid support, or agglutination assay.

Examples of said assays are the sAPPα ELISA kit or the sAPPβ ELISA kit purchased by IBL or by Meso Scale Discovery. Another example of said assays is the multiplex sAPPα and sAPPβ detection kit purchased by Meso Scale Discovery.

In this embodiment, the quantification of sAPPα and/or sAPPβ in a sample is achieved by
contacting said sample with two antibodies that bind to sAPPα or sAPPβ,
measuring the amount of bound anti-sAPPα or anti-sAPPβ antibody and
calculating the amount of or sAPPα or sAPPβ in the sample.

In one embodiment of the invention, the first antibody that binds to sAPPα is an antibody that binds specifically to sAPPα and the second antibody is an antibody directed to an epitope in the N-terminal domain of sAPPα.

In one embodiment of the invention, the first antibody that binds to sAPPβ is an antibody that binds specifically to sAPPβ and the second antibody is an antibody directed to an epitope in the N-terminal domain of sAPPβ.

In one embodiment, a one-step assay (simultaneous incubation of the two antibodies that bind to sAPPα or sAPPβ) is useful. In another embodiment, a two-step assay (sequential incubation of the two antibodies that bind to sAPPα or sAPPβ) is useful. A two-step assay is preferred in the case where other molecules could compete for binding to the antibodies that bind to sAPPα or sAPPβ.

In one embodiment, one antibody that binds to sAPPα or sAPPβ is the “capture” antibody, and is bound to a solid support, such as protein coupling or protein binding surface, colloid metal particles, iron oxide particles, or polymeric beads. One example of polymeric beads is a latex particle. In such an embodiment, the capture antibody is bound to or coated on a solid phase support using standard non-covalent or covalent binding methods, depending on the required analytical and/or solid phase separation requirements. The solid support may be in the form of test-tubes, beads, microparticles, filter paper, membrane, glass filters, magnetic particles, glass or silicon chips or other materials known in the art. The use of microparticles, particularly magnetisable particles, that have been directly coated with the antibody or particles that have been labelled with a universal binder (such as avidin or anti-species antibody) is useful for significantly shortening the assay incubation time.

Alternatively, the capture anti-sAPPα or sAPPβ antibody may be coupled with a ligand that is recognised by an additional antibody that is bound to or coated on the solid support. Binding of the capture antibody to the additional antibody via the ligand then indirectly immobilizes the capture antibody on the support. An example of such a ligand is fluorescein. Alternatively, the binding partner may also be detected indirectly by a secondary detection system. Said secondary detection system is based on several different principles known in the art such as antibody recognition and other forms of immunological or non-immunological bridging and signal amplification detection systems (for example, the biotin-streptavidin system). When a signal amplification system is used, the label includes a first protein such as biotin coupled with the capture antibody, and a second protein such as streptavidin that is coupled with an enzyme. The second protein binds to the first protein. The enzyme produces a detectable signal when provided with substrate(s), so that the amount of signal measured corresponds to the amount of binding partner that is bound sAPPα or sAPPβ. Examples of enzymes include, without limitation, alkaline phosphatase, amylase, luciferase, catalase, beta-galactosidase, glucose oxidase, glucose-6-phosphate dehydrogenase, hexokinase, horseradish peroxidase, lactamase, urease and malate dehydrogenase. Suitable substrates include, without limitation, TMB (3,3′,5,5′-tetramethyl benzidine), OPD (o-phenylene diamine), and ABTS (2,2′-azino-bis(3-ethylbenzthiozoline-6-sulfonic acid). The signal amplification approach may be used to significantly increase the assay sensitivity and low level reproducibility and performance.

Antibodies useful in the various embodiments of the invention encompass commercially available antibodies and antibody fragments, as well as any novel antibodies generated to bind to a suitable epitope on sAPPα or sAPPβ. The antibodies used in various embodiments exemplified herein are monoclonal or polyclonal in nature. Other antibodies and antibody fragments, such as recombinant antibodies, chimeric antibodies, humanised antibodies, Fab or Fv fragments are also useful.

In another embodiment of the invention, the quantification of sAPPα and/or sAPPβ in a sample may be achieved by homogeneous time resolved fluorescence (HTRF).

In one embodiment, a first antibody directed to an epitope in the N-terminal domain of sAPPα or sAPPβ is coupled with a donor fluorophore, such as Europium cryptate (Eu3+ cryptate) or Lumi4™-Tb (Tb2+ cryptate), and a second antibody directed to the cleavage site of sAPPα or sAPPβ is coupled with an acceptor such as XL665, a modified allophycocyanin, or D2 which represents a second generation of acceptor characterized by an organic structure 100 times smaller.

In said embodiment, said first antibody may be the 22C11 clone (ref MAB348 Chemicon) and said second antibody may be the 6E10 clone (ref MAB1560 Chemicon) or the 2B3 antibody (IBL).

In an alternative embodiment, the antibody directed to an epitope in the N-terminal domain of sAPPα or sAPPβ is coupled with an acceptor and the antibody directed to the cleavage site of sAPPα or sAPPβ is coupled with a donor fluorophore.

When these two fluorophores are brought together by a biomolecular interaction, i.e. interaction between antibodies anti-sAPPα or anti-sAPPβ and sAPPα or sAPPβ present in the sample, a portion of the energy captured by the donor fluorophore during excitation is released through fluorescence emission at 620 nm, while the remaining energy is transfered to the acceptor. This energy is then released by the acceptor as specific fluorescence at 665 nm.

Another object of the invention is a method for diagnosing and/or monitoring a neurodegenerative disorder in a subject in need thereof, said method comprising quantifying sAPPα and/or sAPPβ in a sample obtained from said subject, as described here above.

In one embodiment, said neurodegenerative disorder is Alzheimer's disease, early onset familial Alzheimer's disease, amyotrophic lateral sclerosis (Lou Gehrig's disease), Binswanger's disease, corticobasal degeneration (CBD), dementia lacking distinctive histopathology (DLDL), frontotemporal dementia (FTD), Huntington's chorea, multiple sclerosis, myasthenia gravis, Parkinson's disease, trisomy 21 and progressive supranuclear palsy (PSP).

In another embodiment of the invention, said neurodegenerative disorder is Alzheimer's disease.

In another embodiment of the invention, said neurodegenerative disorder is an early onset Alzheimer's disease.

According to the invention, said method for diagnosing and/or monitoring a neurodegenerative disorder in a subject in need thereof, comprises the steps of:
providing a sample obtained from a subject,
treating said sample with a disulfide bonds reducing agent,
measuring sAPPα and/or sAPPβ in said sample by immunological detection,
quantifying the amount of sAPPα and/or sAPPβ present in the sample,
correlating said amount of sAPPα and/or sAPPβ with the diagnosis and/or the monitoring of a neurodegenerative disorder in said subject.

In one embodiment of the invention, measuring sAPPα and/or sAPPβ in said sample by immunological detection comprises:
contacting said treated sample with an antibody that binds specifically to sAPPα or sAPPβ as defined here above under conditions appropriate for formation of a complex between said antibody and sAPPα or sAPPβ present in the sample,
   thereby quantifying the amount of complexes formed to determine the amount of sAPPα and/or sAPPβ present in the sample, and correlating said amount of sAPPα and/or sAPPβ with the diagnosis and/or the monitoring of a neurodegenerative disorder in said subject.

The amount of sAPPα and/or sAPPβ quantified may thus be compared with the corresponding amount detected in the samples of control subjects, in previous samples obtained from the subject or with normal reference values.

While the method of the invention is intended for the diagnosis of a neurodegenerative disorder, control subjects are for example subjects that have not been diagnosed for said neurodegenerative disorder. Normal reference values refer to the amount of sAPPα and/or sAPPβ that can be determined by the method of the invention in a subject that has not been diagnosed for a neurodegenerative disorder.

In one embodiment of the invention, said control value or reference value is determined by using the average values obtained from at least 10, preferably from at least 100 control subjects.

Quantifying the amount of sAPPα and/or sAPPβ is also of interest for monitoring for example a therapeutic treatment against said neurodegenerative disorders, such as for example a treatment with NSAID (MPC-7869, R-flurbiprofen) or statins.

Another object of the invention is a kit for use in the method of the invention as described here above, said kit comprising, as separate components,
a disulfide bonds reducing agent, and
at least one antibody that binds to sAPPα or sAPPβ.

In one embodiment, said antibody binds specifically to sAPPα or sAPPβ. Said antibody is for example an antibody that binds on the cleavage site of sAPPα or sAPPβ.

Examples of said antibody include, but are not limited to, the monoclonal antibody 6E10, the monoclonal antibody 2B3 and the rabbit antibody 3329 (PMID 9465092), the specific sAPPβ antibody described in WO2008/008463.

In another embodiment, said kit comprises two antibodies that bind to sAPPα or sAPPβ. Among these two antibodies, one binds specifically to sAPPα or sAPPβ and the other is directed to an epitope in the N-terminal domain of sAPPα or sAPPβ.

Examples of said antibody directed to an epitope in the N-terminal domain of sAPPα or sAPPβ include, but are not limited to, the monoclonal antibody BAN50 (PMID: 10480887), the monoclonal antibody 22C11, the polyclonal antibody polyC11 (Upstate/Millipore, Cat AB5368, Chemicon), the polyclonal antibody sAPP from OYC (Cat APP-KPI-Antiserum), the polyclonal antibody sAPP from Signet Covance (Cat SIG-39139), the monoclonal antibody 1E8 (U.S. Pat. No. 6,849,416).

In one embodiment, one antibody may be coated to a solid support. Suitable examples of solid support are identified here above.

In another embodiment, one or more of the antibodies may be labelled. Suitable examples of labels are similarly identified here above.

The kit may also contain optional additional components for performing the method of the invention. Such optional components are for example containers, mixers, buffers, instructions for assay performance, labels, supports, and reagents necessary to couple the antibody to the support or label.

The invention relates to a method for identifying a molecule that modulates the alpha-secretase activity and/or beta-secretase activity, comprising:

a) providing, in a suitable media, cells showing a beta-secretase and/or alpha-secretase activity, and expressing a β-amyloid precursor protein (APP);

b) contacting the cells with a candidate compound; and

c) quantifying sAPPα and/or sAPPβ according to the method as described hereabove.

In said method, a change in the level of sAPPα and/or sAPPβ, compared to control cells, indicates whether the candidate compound modulates the alpha-secretase activity and/or beta-secretase activity.

In one embodiment, said cells showing a beta-secretase and/or alpha-secretase activity, and expressing a β-amyloid precursor protein (APP) are human neuroblastoma cells differentiated in the presence of retinoic acid during 1 week.

In another embodiment, said cells showing a beta-secretase and/or alpha-secretase activity, and expressing a β-amyloid precursor protein (APP) are primary neurons (isolated from cortex and/or hippocampus) from transgenic mice overexpressing wild-type APP.

In another embodiment, said cells showing a beta-secretase and/or alpha-secretase activity, and expressing a β-amyloid precursor protein (APP) are a cell line transfected with constructs to show a beta-secretase and/or alpha-secretase activity and with a construct encoding for APP.

In one embodiment of the invention, the cells are contacted with the candidate compound during 12 to 36 h, preferably during 20 h to 28 h before quantifying sAPPα and/or sAPPβ.

As used herein, “modulating the alpha-secretase activity and/or beta-secretase activity” refers to the ability of a molecule to change, i.e. inhibit/decrase or increase, alpha-secretase and/or beta-secretase cleavage of APP in cells. In one embodiment, the molecule is capable of inhibiting or decreasing the build up of sAPPβ. In another embodiment, the molecule is capable of increasing the build up of sAPPα.

In one embodiment, said molecule capable of modulating the alpha-secretase activity and/or beta-secretase activity is identified in large scale screens. Such screens include screens wherein hundreds or thousands or more of candidate compounds are screened in a high-throughput format for alpha-secretase activity and/or beta-secretase activity modulators.

As used herein, “candidate compound” encompass numerous chemical classes, although typically they are organic molecules, preferably small organic compounds having a molecular weight of more than 50 and less than about 2,500 daltons. Candidate compounds comprise functional groups necessary for structural interaction with proteins, particularly hydrogen bonding, and typically include at least an amine, carbonyl, hydroxyl or carboxyl group, preferably at least two of these functional chemical groups. The candidate compounds may also comprise cyclical carbon or heterocyclic structures and/or aromatic or polyaromatic structures substituted with one or more of the above functional groups. Candidate compounds are also found among biomolecules including but not limited to peptides, saccharides, fatty acids, steroids, purines, pyrimidines, derivatives, structural analogs or combinations thereof.

Candidate compounds are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

The method of the invention thus provide convenient means to detect and/or quantify the alpha-secretase activity and/or beta-secretase activity and identify candidate compounds that are alpha-secretase activity and/or beta-secretase activity modulators, and use such compounds to treat, prevent or alleviate the symptoms of a neurodegenerative disease. The screening method of the invention provide a powerful platform on which small molecule and RNAi screening (both conventional and high-throughput) can take place to identify potential therapeutic candidates (candidate compounds).

The following examples are given for the purpose of illustrating various embodiments of the invention.

### BRIEF DESCRIPTION OF THE FIGURES

FIG. 1: Dimerization of sAPPα through disulfide bonds in different samples

A, B, C: western blot after migration on NU-PAGE gels (4-12%).

A: 1-2: recombinant sAPPα produced by bacteria and loaded with (+) or without (−) DTT

3-4: recombinant sAPP-Fc produced by COS cells and loaded with (+) or without (−) DTT

B: conditioned medium of human neuroblastoma cells SH-SY5Y, treated with PdBu (increase the alpha secretase activity) and loaded with (+) or without (−) DTT.

C: CSF with (+) or without (−) DTT

FIG. 2: Dimerization of bacterial recombinant sAPP beta

1: in absence of DTT

2: in presence of DTT

FIG. 3: Comparison of samples treated by heating or DTT

A: human serum samples (7) and B: human CSF samples (7)

FIG. 4: Specificity of the test for sAPPα using recombinant sAPPα, sAPPβ and AB 1-42.

FIG. 5: Linearity of the dose-dependence response both for recombinant sAPPα, human serum, human CSF and conditioned medium of neuroblastoma cell line

FIG. 6: Comparison of HTRF test with ELISA kits (IBL) in presence or absence of DTT

FIG. 7: sAPPα in SH-SY5Y neuroblastoma cell line conditioned medium media using HTRF test

FIG. 8: sAPPα in conditioned medium from primary mouse cortical neurons using HTRF test

### EXAMPLES

### Experimental Procedures

HTRF Assay Method

Standard Curve:

Recombinant sAPPα was produced in bacteria, using a p-Gex vector and purified from bacterial lysates, in one step, using the Glutathione —S-transferase system (GE Healthcare).

Concentration range: from 3 to 200 ng/ml which corresponds to 0.015 to 1 ng by well.

Samples:

Four types of biological samples were investigated using the following procedure: human CSF, human blood serum, culture medium of neuroblastoma SH-SY5Y cell line treated with different drugs (for instance the PKC activator PdBu which stimulate the ADAM 17 alpha secretase activity), or conditioned culture medium from primary mouse cortical neurons.

Procedure:

All samples and calibrator were treated with 10 mM DTT on ice, during 30 min. In a 384-well low volume (20 μl) white plate, reagents were distributed in the following order:
5 μl 150 mM phosphate buffer pH 7.0, 0.2% BSA.
5 μl sample or calibrator, treated with DTT and diluted.
5 μl MAB348 antibody, cryptate conjugate, diluted 1/250 with 50 mM phosphate buffer pH 7.0, 0.4M KF.
5 μl 2B3 antibody, D2 conjugate, diluted 1/250 with 50 mM phosphate buffer pH 7.0, 0.4M KF.

For the negative control, the calibrator was replaced by 5 μl of buffer.

The plate covered with a plate sealer was centrifuged (1000 g 1 min) and incubated overnight, at room temperature.

The plate sealer was removed, and the plate read on a compatible HTRF reader (Envision, Perkin Elmer), at 620 and 665 nM.

Data Evaluation:

Results are calculated from 665 nm/620 nm ratio and expressed in delta F:

$$delta F = \frac{\begin{matrix}sample or calibrator 665 nm \text{/} 620 nm ratio - \\ negative control 665 nm \text{/} 620 nm ratio\end{matrix}}{negative control 665 nm \text{/} 620 nm ratio}$$

Delta F is proportional to the sAPPα concentration; Delta F obtained for samples can be reported on the calibration curve to deduce respective sAPPα concentration.

Results

sAPPα and sAPPβ are Dimerized by Disulfides Bonds.

A recombinant sAPPα without any Tag and a recombinant sAPPα-Fc were constructed and produced in bacteria.

sAPPα was detected in a western-blot assay using the 6E10 antibody, specific of the C-terminal sequence of the sAPPα.

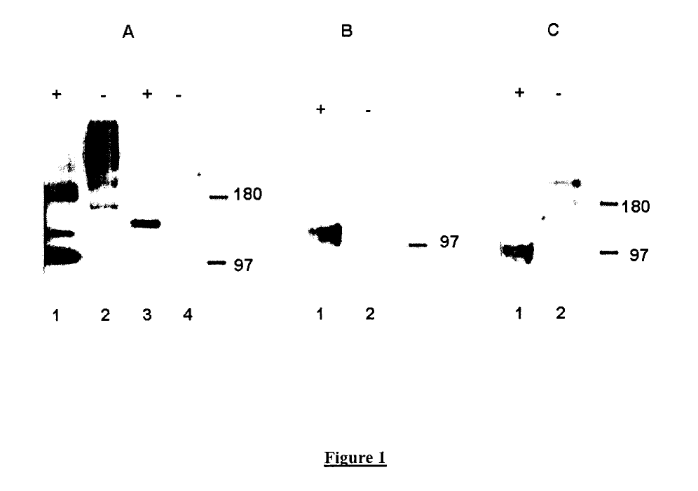

FIG. 1A shows
lanes 1-2: recombinant sAPPα produced by bacteria and incubated with (+) or without (−) DTT for 10 nm before loading;
lanes 3-4: recombinant sAPP-Fc produced by COS cells and incubated − with (+) or without (−) DTT for 10 nm.

5 ng of sAPPα protein were loaded per well.

FIG. 1A shows that in the absence of DTT, sAPPα is detected as di or trimers suggesting that the protein can homodimerize or oligomerize by disulfide bridges. In presence of DTT recombinant sAPPα produced in bacteria is expected at 100 kDa (lane 1) and recombinant sAPP-Fc is expected at 130 kDa (lane 3).

In FIG. 1B, conditioned medium of human neuroblastoma cells SH-SY5Y was treated with PdBu (an agent that increases the alpha secretase activity) and incubated for 10 nm with DTT (+) or not (−) before loading.

In FIG. 1C, cerebrospinal fluid samples (CSF) were incubated for 10 nm with DTT (+) or in absence (−) of DTT before loading.

FIG. 1B and FIG. 1C show that, in the conditioned medium of human neuroblastoma cells SH-SY5Y, (FIG. 1B) or in human CSF (FIG. 1C), sAPPα is correctly detected only in the presence of DTT suggesting the presence of disulfide bridges.

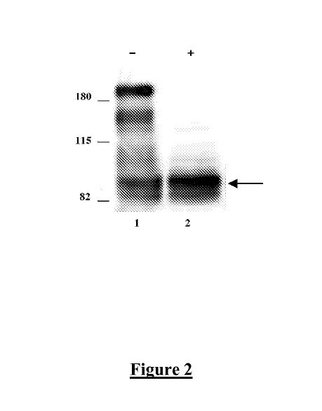

FIG. 2 shows:
lane 1: recombinant sAPPβ produced in bacteria and incubated without DTT (−)
lane 2: recombinant sAPPβ produced in bacteria and incubated with DTT (+)

FIG. 2 shows in lane 1 a major band around 200 kDa while after incubation with DTT (+) for 10 min, a band at 97 kDa is observed (lane 2).

This result suggests that sAPPβ homodimerize or oligomerize by disulfide bridges.

Comparison of Thermal Pre-Treatment and DTT Pre-Treatment.

1) Comparison on Human Blood Serum Samples

Serum samples were treated, either with 10 mM DTT on ice during 30 min (2 and 5), or by heating at 66° C. during 10 min (4), or both heating at 66° C. and DTT (6). Control samples were not submitted to any treatment (1 and 3).

Two different buffers were tested, 50 mM phosphate buffer pH 7.4, 0.2% BSA (1-2), and 50 mM saline phosphate buffer pH 7.4, 5% BSA, Tween 20 0.05% (3-6).

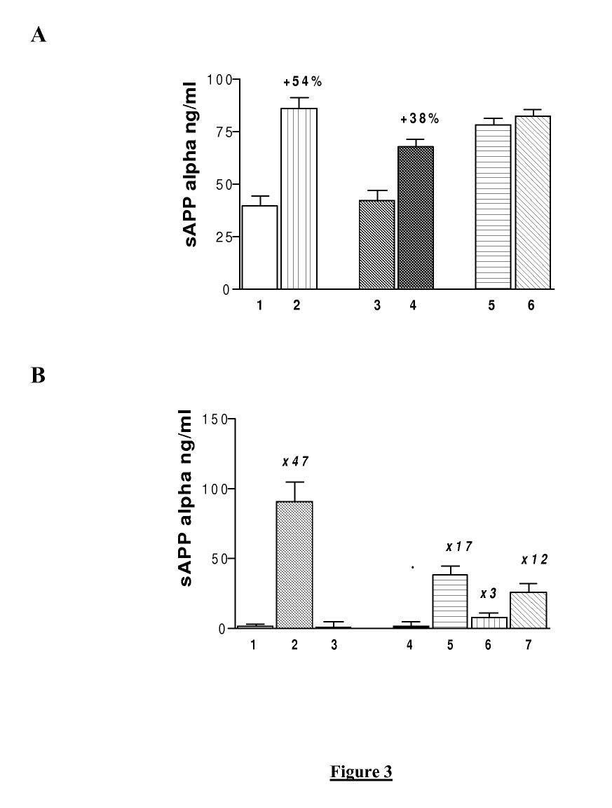

Next, sAPPα was measured in each sample with the HTRF method described above. FIG. 3A shows that DTT treatment induces a 54% increase of sAPPα (2), compared to control (1), while heating induces only a 38% increase (4) compared to control (3). Heating combined to DTT treatment (6) does not allow a sAPPα recovery beyond the DTT treatment alone (5).

2) Comparison on Human Cerebrospinal Fluid Samples

CSF samples were treated, either with 10 mM DTT on ice during 30 min (2 and 5), or by heating at 66° C. during 10 min (3 and 6), or both at 66° C. and DTT (7). Control samples were not submitted to any treatment (1 and 4).

Two different buffers were tested, 50 mM phosphate buffer pH 7.4, 0.2% BSA (1-3), and 50 mM saline phosphate buffer pH 7.4, 5% BSA, Tween 20 0.05% (4-7).

Next, sAPPα was measured in each sample with the HTRF method described above. FIG. 3B shows that DTT treatment induces a strong increase (×47 or ×17) of sAPPα (2 and 5), compared to control (1 and 4)), while heating induces no effect (3) or only a slight effect (6) compared to control (1 and 4). Heating combined with DTT treatment (7) does not allow a sAPPα recovery beyond the DTT treatment alone (5).

Absolute Requirement of DTT Pre-Treatment.

Different samples were submitted to the sAPPα HTRF assay with or without DTT treatment. Results are shown in Table 1.

TABLE 1

| | With DTT | Without DTT |
|---|---|---|
| Samples | sAPP alpha ng/ml | sAPP alpha ng/ml |
| CSF (8) | 101.6 ± 19.4 | 3.0 ± 0.7 |
| Serum (8) | 87.8 ± 5.7 | 26.9 ± 2.1 |
| Conditioned medium control (8) | 29.8 ± 2.0 | 3.1 ± 0.5 |
| Conditioned medium PdBu (8) | 59.1 ± 2.8 | 3.4 ± 0.4 |

In the absence of DTT, no detection of sAPPα is observed in human CSF or conditioned medium of neuroblastoma cell line.

The addition of PdBu in conditioned medium of neuroblastoma cell line activates the production of sAPPα. This increase in the amount of sAPPα can only be seen in samples pre-treated with DTT, while sAPPα was not detected in sample not pre-treated with DTT.

However, contrary to the CSF, it is possible to detect a small amount of the sAPPα in the serum under non reducing conditions, suggesting that native sAPPα in blood could be present under monomers and oligomers while this is not the case in CSF where the major part is under dimers.

Performance Characteristics of the HTRF Test.

1) Sensitivity

Detection limit is <3 ng/ml that is to say 15 pg per well

2) Specificity

Recombinant proteins sAPPα and sAPPβ were produced in bacteria by the glutathione-S-transferase method (GE Healthcare). Aβ 1-42 peptide is commercially available.

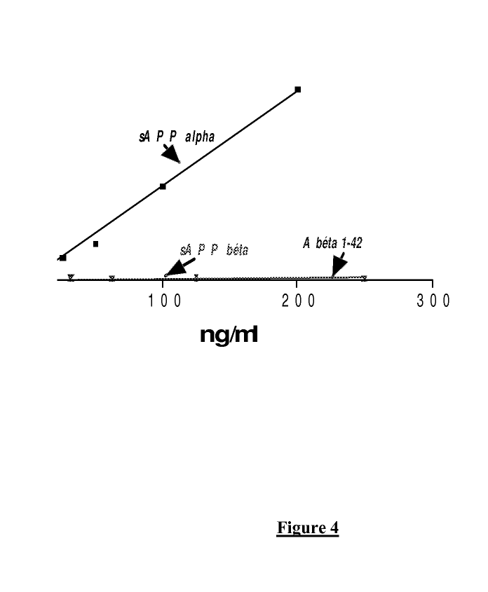

The assay is specific for sAPPα. FIG. 4 shows that no cross reactivity is observed with sAPPβ or Aβ peptide amyloid 1-42 at concentrations up to 1000 ng/ml

3) Intra Assay Reproducibility (ng/ml)

Table 2 shows the intra assay reproducibility.

TABLE 2

| Sample | Mean | SD value | Error % | Sample number |
|---|---|---|---|---|
| CSF 1 | 75.0 | 1.8 | 2.5 | 8 |
| CSF 2 | 37.5 | 0.7 | 1.9 | 8 |
| Serum 1 | 18.5 | 0.4 | 2.1 | 8 |
| Serum 2 | 29.4 | 0.6 | 2.2 | 8 |
| Conditioned | 29.8 | 2.0 | 6.7 | 8 |
| medium control | | | | |
| Conditioned | 59.1 | 2.7 | 4.5 | 8 |
| medium PdBu | | | | |

4) Inter Assay Reproducibility (ng/ml)

Table 3 shows the inter assay reproducibility.

TABLE 3

| Sample | Mean | SD value | Error % | Sample number |
|---|---|---|---|---|
| CSF 1 | 80.1 | 2.7 | 3.3 | 12 |
| CSF 2 | 41.2 | 1.6 | 3.8 | 12 |
| Serum 1 | 20.4 | 0.8 | 3.9 | 12 |
| Serum 2 | 31.4 | 0.9 | 2.8 | 12 |
| Conditioned | 29.7 | 2.0 | 6.7 | 16 |
| medium control | | | | |
| Conditioned | 61.4 | 3.6 | 5.8 | 16 |
| medium PdBu | | | | |

5) Added Recovery Assay

The measured values of added recombinant sAPPα (theoretical value) to CSF or serum, or conditioned medium are in a recovery from 70 to 119% (see table 4) showing that there is no interference with the matrix

TABLE 4

| | Theoretical | | | |
|---|---|---|---|---|
| | value | Measurement | Recovery | |
| Sample | ng/ml | value ng/ml | % | Sample number |
| CSF 1 | 15 | 10.8 ± 0.8 | 72 | 11 |
| | 30 | 30.4 ± 1.6 | 100 | |
| CSF 2 | 15 | 13.8 ± 0.6 | 92 | 12 |
| | 30 | 35.8 ± 0.6 | 119 | |
| Serum 1 | 15 | 14.6 ± 0.8 | 97 | 12 |
| | 30 | 24.3 ± 1.3 | 81 | |
| Serum 2 | 15 | 10.6 ± 0.7 | 70 | 11 |
| | 30 | 20.3 ± 0.5 | 67 | |
| Conditioned | 15 | 15.4 ± 0.4 | 102 | 8 |
| medium | 30 | 31.5 ± 0.6 | 104 | |

6) Linearity

CSF samples were diluted 1/2, 1/4, 1/8, 1/16. Serum and conditioned medium were diluted 1/2, 1/4, 1/8.

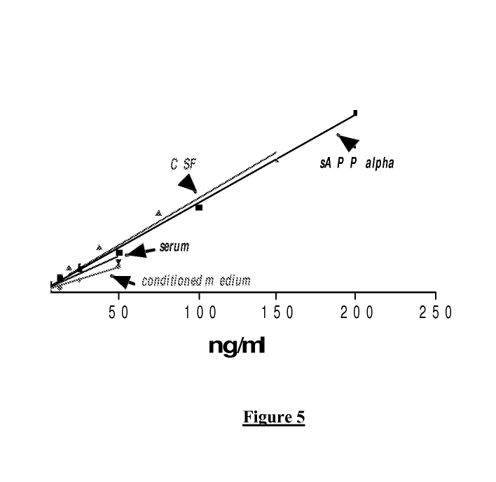

FIG. 5 shows a linearity of the values at different concentrations of the recombinant sAPPβ alpha protein. Same linearity was observed using different dilutions of serum, CSF and conditioned media samples.

Comparison of ELISA/HTRF Results.

CSF were tested using 3 different protocols:
1 Using the instructions of the ELISA commercialized by the IBL company, that is to say without any samples pretreatment.
2 Using the same ELISA procedure, but with a sample pre-treatment with 10 mM DTT.
3 Using the HTRF procedure described above, with a sample pre-treatment with 10 mM DTT

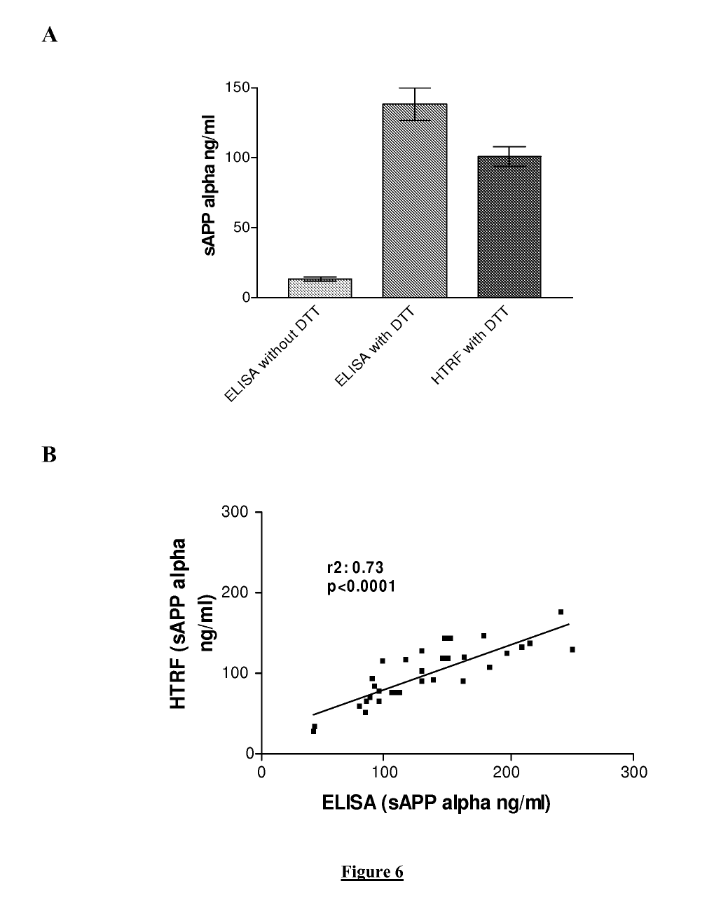

Using the commercially available ELISA assay, we found 20 fold higher values, when samples were treated with DTT compared to no treated samples (FIG. 6A). In addition, the results obtained with the HTRF method were similar and closely related (FIGS. 6A and 6B) to that found with the ELISA on the same treated samples.

Validation of the sAPPα HTRF Assay in SH-SY5Y Neuroblastoma Cells Conditioned Medium.

Numerous studies have shown that the sAPPα secretion is regulated by some intracellular signalisation pathway involving PKC and that the α-secretase responsible for this secretion is a disintegrin-metalloproteinase (ADAM 17).

A way to validate at the same time, the HTRF assay and the cellular test, was to show that we obtained an increase of the sAPPα secretion when the PKC was activated by a phorbol ester (PdBu), and that this effect was abolished by a PKC inhibitor (GF 109203) and by an alpha secretase inhibitor (TAPI 0).

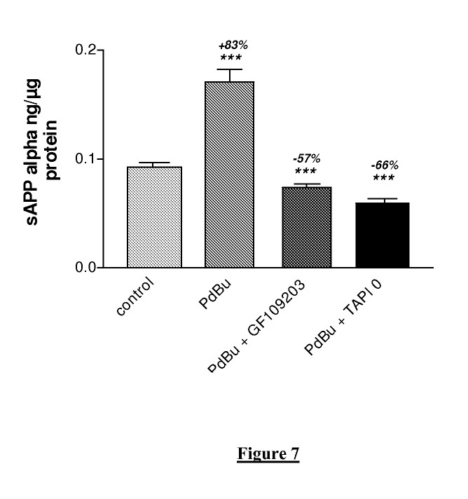

FIG. 7 shows that the amount of sAPPα is increased in the presence of PdBu and decreased in the presence of a PKC inhibitor or an alpha secretase inhibitor.

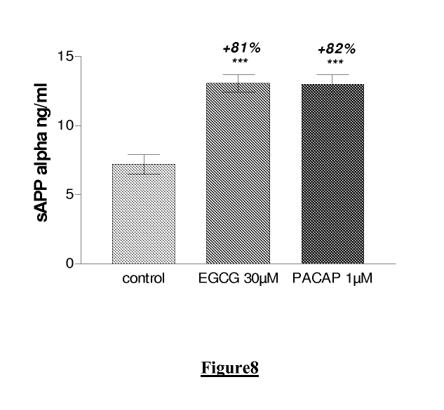

Validation of the sAPPα HTRF Assay in Primary Cortical Neurons Conditioned Medium. (FIG. 8)

In the same way, it is possible to show an increase of sAPP alpha in the medium of primary neurons from transgenic mice expressing wild type human APP, after the addition of drugs like PACAP 1-27 or EGCG, already known to stimulate alpha secretase activity.

1. A method for detecting or quantifying the presence of sAPPα and/or sAPPβ in a sample, said method comprising:
treating the sample with a disulfide bonds reducing agent,
measuring sAPPα and/or sAPPβ in said sample by immunological detection.

2. The method according to claim 1, wherein said disulfide bonds reducing agent is 2-mercaptoethanol (ME), dithiothreitol (DTT), tris(2-carboxyethyl)phosphine (TCEP) or a combination thereof.

3. The method according to claim 1, wherein said sample is a culture medium sample, a serum sample, a plasma sample, a cerebrospinal fluid sample, or a brain tissue sample.

4. The method according to claim 1, wherein the immunological detection of sAPPα and/or sAPPβ is carried out by using at least one antibody that binds specifically to sAPPα or sAPPβ.

5. The method according to claim 1, wherein a second antibody that binds to an epitope in the N-terminal domain of sAPPα or sAPPβ is used.

6. The method according to claim 1, wherein the immunological detection of sAPPα and/or sAPPβ is carried out by an enzyme immunoassay or enzyme-linked immunoassay (EIA or ELISA).

7. The method according to claim 1, wherein the immunological detection of sAPPα and/or sAPPβ is carried out by homogeneous time resolved fluorescence (HTRF).

8. A method for determining and/or monitoring a neurodegenerative disorder in a subject in need thereof, said method comprising quantifying sAPPα and/or sAPPβ in a sample obtained from said subject according to the method of claim 1.

9. The method according to claim 8, wherein said method is for diagnosing and/or monitoring a neurodegenerative disorder, said neurodegenerative disorder being Alzheimer's disease, early onset familial Alzheimer's disease, amyotrophic lateral sclerosis (Lou Gehrig's disease), Binswanger's disease, corticobasal degeneration (CBD), dementia lacking distinctive histopathology (DLDL), frontotemporal dementia (FTD), Huntington's chorea, multiple sclerosis, myasthenia gravis, Parkinson's disease, trisomy 21 or progressive supranuclear palsy (PSP).

10. The method according to claim 9, wherein said method is for diagnosing and/or monitoring Alzheimer's disease.

11. The method according to claim 10, wherein said method is for diagnosing and/or monitoring early onset familial Alzheimer's disease.

12. A kit for use in the method according to claim 8, comprising as separate components:
a disulfide bonds reducing agent, and
at least one antibody that binds specifically to sAPPα or sAPPβ.

13. The kit according to claim 12, further comprising a second antibody that binds to an epitope in the N-terminal domain of sAPPα or sAPPβ.

14. The kit according to claim 12, wherein at least one antibody that binds to sAPPα or sAPPβ is coated to a solid support and/or at least one antibody that binds to sAPPα or sAPPβ is labelled.

15. A method for identifying a molecule that modulates the alpha-secretase activity and/or beta-secretase activity, comprising:
a) providing, in a suitable media, cells showing a beta-secretase and/or alpha-secretase activity, and expressing β-amyloid precursor protein (APP);
b) contacting the cells with a candidate compound; and
c) quantifying sAPPα and/or sAPPβ according to the method of claim 1.
